(19) United States
(12) Patent Application Publication
(10) Pub. No.: US 2025/0134232 A1
(43) Pub. Date: May 1, 2025

(54) ARTIFICIAL NAIL

(71) Applicant: Guiwen Cai, Guangzhou (CN)

(72) Inventor: Guiwen Cai, Guangzhou (CN)

(21) Appl. No.: 18/988,986

(22) Filed: Dec. 20, 2024

Related U.S. Application Data
(63) Continuation of application No. 18/401,543, filed on Dec. 31, 2023, now Pat. No. 12,207,720.

(30) Foreign Application Priority Data
Oct. 27, 2023 (CN) 202322904759.X

(51) Int. Cl. A45D 31/00

(52) U.S. Cl. CPC A45D 31/00; A45D 2031/005

(57) ABSTRACT

An artificial nail has a top surface, a portion of the top surface is recessed inwardly to form a plurality of guiding grooves, and the plurality of guiding grooves are used for guiding a user to draw a pattern or mount a decorative piece on the top surface of the artificial nail. The artificial nail comprises: a first edge configured to be disposed near a natural cuticle of the real nail; a second edge opposite to the first edge; a third edge and a fourth edge that are connected between the first edge and the second edge and are spaced apart from each other. The plurality of guiding grooves comprise a first curved groove and a second curved groove; each of the first curved groove and the second curved groove has one end disposed near the third edge and the other end disposed near the fourth edge.

### CROSS REFERENCE TO RELATED APPLICATIONS

The present application is a continuation application of the U.S. patent application Ser. No. 18/401,543, filed on Dec. 31, 2023, which claims the priority of the Chinese patent application No. CN202322904759.X, filed on Oct. 27, 2023. Contents of which are incorporated herein by its entireties.

### TECHNICAL FIELD OF THE INVENTION

The present disclosure relates to the technical field of beautifying nails, in particular to an artificial nail.

### BACKGROUND OF THE INVENTION

Artificial nails, also known as false nails, fashion nails, acrylic nails, nail covers or nail extensions, are extensions placed on a person's nails as fashion accessories.

In order to facilitate artistic aesthetics, guide lines for guiding people to draw patterns or install decorative parts are generally provided on the surface of the artificial nails, but, in practice, the guide lines of the line structure are easily polished off in the polishing step of the artificial nails, so that the people cannot be guided to draw patterns or install decorative parts on the artificial nails, and in the absence of polishing, the nail varnish applied to the artificial nails cannot be reliably bonded with the artificial nails, resulting in the easy peeling off of the nail varnish layer and the difficulty in covering the guide lines of the line structure by the sealer, thus affecting the aesthetic appearance.

### SUMMARY OF THE INVENTION

The present disclosure provides an artificial nail, configured to be placed on a real nail of a user. The artificial nail has a top surface, a portion of the top surface is recessed inwardly to form a plurality of guiding grooves, and the plurality of guiding grooves are used for guiding a user to draw a pattern or mount a decorative piece on the top surface of the artificial nail. The artificial nail comprises: a first edge configured to be disposed near a natural cuticle of the real nail; a second edge opposite to the first edge; a third edge and a fourth edge that are connected between the first edge and the second edge and are spaced apart from each other. The plurality of guiding grooves comprise a first curved groove and a second curved groove; each of the first curved groove and the second curved groove has one end disposed near the third edge and the other end disposed near the fourth edge; each of the first curved groove and the second curved groove is symmetrical about a centerline of the artificial nail; and the first curved groove has no intersection with the second curved groove.

### BRIEF DESCRIPTION OF THE DRAWINGS

In the accompanying drawings, unless otherwise specified, the same reference signs in the accompanying drawings indicate the same or similar parts or elements. These accompanying drawings are not necessarily drawn to scale. It should be understood that these accompanying drawings depict only some of the embodiments disclosed in accordance with the present disclosure and should not be viewed as limiting the scope of the present disclosure.

FIG. 1 shows a schematic view of the front structure of an artificial nail of the present disclosure;

FIG. 2 shows a schematic view of the front structure of the artificial nail of the present disclosure;

FIG. 3 shows a schematic view of the front structure of the artificial nail of the present disclosure;

FIG. 4 shows a schematic view of the reverse side structure of the artificial nail of the present disclosure.

Reference signs: 1, nail flake (i.e. fake fingernail flake); 11, guiding groove; 111, transverse groove; 112, longitudinal groove; 12, glossy layer; 13, second frosted layer; 14, first frosted layer; 15, third frosted layer; 16, colorful translucent coating.

### DETAILED DESCRIPTION OF THE PREFERRED EMBODIMENT

In the following, only certain exemplary embodiments are briefly described. As can be recognized by those skilled in the art, the described embodiments can be modified in a variety of different ways without departing from the spirit or scope of the present disclosure. Accordingly, the accompanying drawings and description are considered to be essentially exemplary and not limiting.

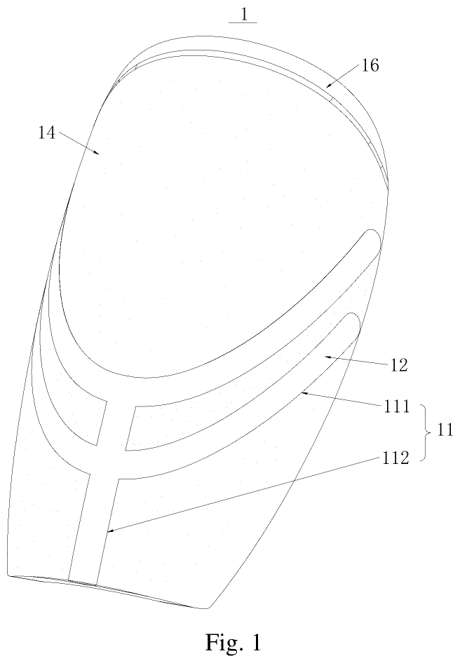

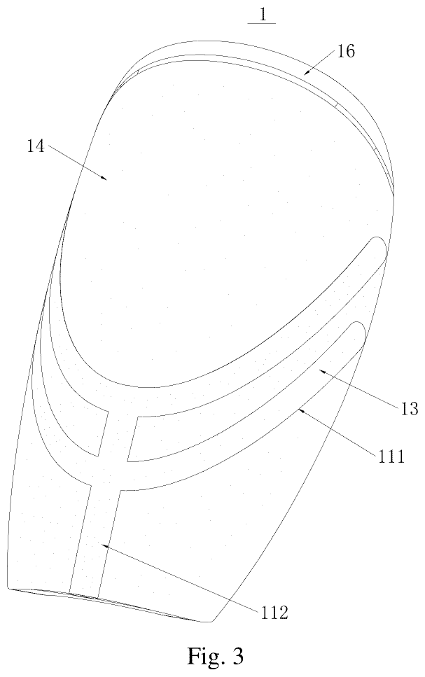

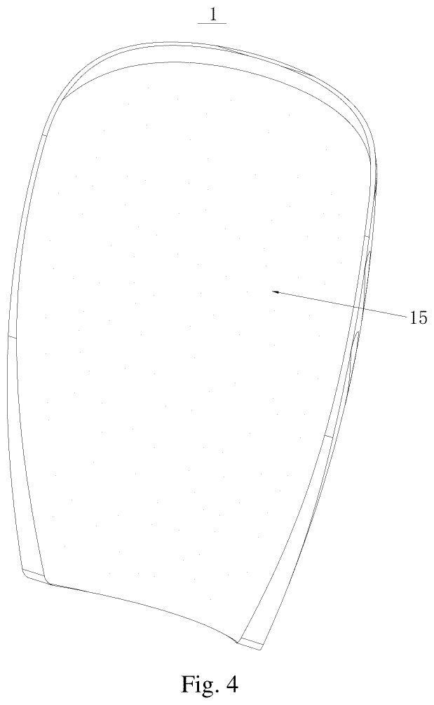

Referring to FIGS. 1-FIG. 4, there is shown an artificial nail of a preferred embodiment of the present disclosure, the artificial nail comprises a nail flake 1, a guiding groove 11 is provided on the top surface of the nail flake 1, the guiding groove 11 has a groove depth of d, the guiding groove 11 has a groove width of b, with 0.05 mm≤d≤0.2 mm, and 0.05 mm≤b≤0.2 mm, and the guiding groove 11 is used for guiding the user to draw a pattern (such as a French pattern or a line pattern, etc.) or to mount a decorative piece on the top surface of the nail flake 1.

With the artificial nail of the present disclosure, since the guiding groove 11 is provided on the nail flake 1, the guiding groove 11 in the form of a groove has a certain depth and width, and the outline is clearly visible, so that it is easy to guide the user to position the design on the nail flake 1 and to complete the design quickly, for example, to guide the user to draw a pattern or install a decorative piece on the nail flake 1. In addition, the guiding groove 11 in the form of a groove can be filled by the sealing layer applied to the top surface of the nail flake 1 easily, without leaving traces, so that the top surface of the nail flake 1 is smooth and even, and the shape is more beautiful; in addition, the groove 11 in the form of a groove is more resistant to abrasion, and will not be easily polished off, so that the groove 11 can be clearly shown on the top surface of the nail flake 1 even after the top surface of the nail flake 1 is sanded, thus facilitating the guidance of the user to operate according to a set pattern; furthermore, because 0.05 mm≤d≤0.2 mm and 0.05 mm≤b≤0.2 mm, the guiding groove 11 has a suitable depth and width to ensure that the guiding groove 11 is clearly presented on the top surface of the nail flake 1, and at the same time, the guiding groove 11 can be easily filled with less sealing material, which saves material and improves the operation efficiency.

In one embodiment, the groove walls of the guiding groove 11 are glossier than the rest of the top surface of the nail flake 1 so as to highlight the guiding groove 11 and make it easier for the user to observe the direction of the guiding groove 11, thereby making it easier to guide the user to quickly complete the design.

In one embodiment, the rest of the top surface of the nail flake 1 has a matte layer, and the groove wall of the guiding groove 11 has a glossy layer 12. The top surface of the nail flake 1 set matte has a certain roughness, which facilitates a reliable combination of the top surface of the nail flake 1 and the sealing layer, improves the reliability of the connection between the nail flake 1 and the sealing layer, and makes the sealing layer less likely to come off, whereas the guiding groove 11 set glossy facilitates highlighting the contour of the guiding groove 11, thereby making the contour of the guiding groove 11 more clearly visible and facilitating efficient operation of the user.

Specifically, in one embodiment, the aforesaid matte layer is the first frosted layer 14, i.e., all parts of the top surface of the nail flake 1 other than the guiding grooves 11 are frosted surfaces that have undergone frosting treatment, so as to improve the roughness of the remaining parts of the top surface of the nail flake 1, so as to facilitate the reliable bonding of the top surface of the nail flake 1 with the sealing layer, improve the reliability of the connection between the nail flake 1 and the sealing layer, and make it difficult for the sealing layer to fall off.

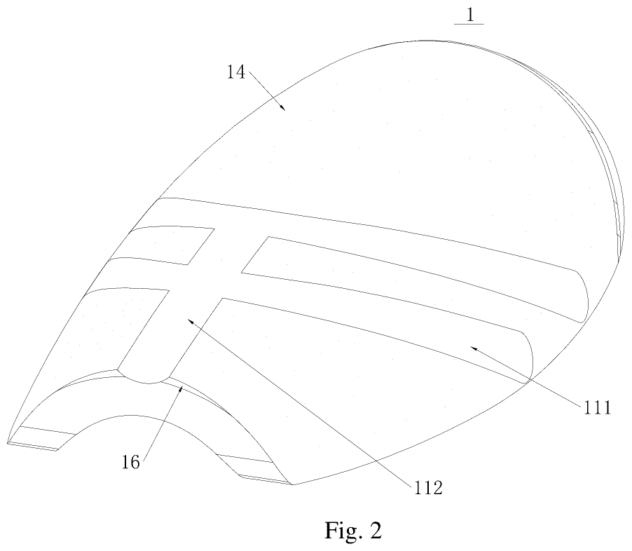

Referring to FIG. 2, in one embodiment, the groove wall of the guiding groove 11 has a second frosted layer 13 to improve the roughness of the groove wall of the guiding groove 11, to facilitate reliable bonding of the sealing layer filled in the guiding groove 11 with the nail flake 1, to improve the connection reliability of the nail flake 1 and the sealing layer, and to make the sealing layer less likely to fall off.

Referring to FIG. 1, in one embodiment, the number of guiding grooves 11 is plurality, at least one of the guiding grooves 11 extends along the length direction of the nail flake 1 (i.e., longitudinal groove 112), and the rest of the guiding grooves 11 extend along the width direction of the nail flake 1 (i.e., transversal grooves 111) and intersect with the other guiding grooves 11 that extend along the length direction of the nail flake 1, so as to make the top surface of the nail flake 1 have a pattern with better appearance that improves the aesthetics of the artificial nail. Of course, in other embodiments, the number, shape, and arrangement of the guiding grooves 11 can be adjusted according to actual needs.

In one embodiment, the bottom surface of the nail flake 1 has a third frosted layer 15 to provide the bottom surface of the nail flake 1 with a certain degree of roughness, so that the nail glue tightly connects the bottom surface of the nail flake 1 to the human nail without applying a primer, when the artificial nail being applied to the human nail, which can simplify the operation process and improve the operation efficiency at the same time.

In one embodiment, the nail flake 1 is a structure made of colored translucent material, which makes the nail flake 1 have a color and a more beautiful appearance, and at the same time, it is also possible to dispense with the need to coat the nail flake 1 with colored nail glue oil when applying the nail flake to a human nail, which simplifies the operation process and also improves the operation efficiency.

Specifically, the above mentioned colored translucent nail flake 1 is injection molded from a uniform mixture of color powder and acrylic material.

In one embodiment, the nail flake 1 is provided with a colored translucent coating 16, so that the nail flake 1 has a color and has a more beautiful appearance, and at the same time, it can also eliminate the need to apply colored nail glue oil when applying the nail flake 1 to a human nail, which can simplify the operation process and at the same time, can also improve the operation efficiency.

In the description of the present specification, the terms “an embodiment”, “some embodiments”, “example”, “specific example”, or “some examples”, etc. mean that the specific features, structures, materials, or characteristics described in connection with the embodiment or example are included in at least one embodiment or example of the present disclosure. Moreover, the specific features, structures, materials, or characteristics described can be combined in any one or more of the embodiments or examples in a suitable manner. Moreover, without contradicting each other, those skilled in the art can connect and combine different embodiments or examples and features of different embodiments or examples described herein.

Furthermore, the terms “first” and “second” are used only for descriptive purposes and are not to be understood as indicating or implying relative importance or implicitly specifying the number of technical features indicated. Thus, a feature defined with the terms “first”, “second” can expressly or impliedly include at least one such feature. In the description of the present disclosure, “more than one” means two or more, unless otherwise expressly and specifically limited.

The above are only specific embodiments of the present disclosure, but the scope of protection of the present disclosure is not limited thereto, and any person skilled in the art can easily think of various variations or substitutions thereof within the scope of the technology disclosed in the present disclosure, all of which shall be covered by the scope of protection of the present disclosure. Therefore, the scope of protection of this application shall be subject to the scope of protection of the claims.

What is claimed is:

1. An artificial nail, configured to be placed on a real nail of a user, wherein,
the artificial nail has a top surface, a portion of the top surface is recessed inwardly to form a plurality of guiding grooves, and the plurality of guiding grooves are used for guiding a user to draw a pattern or mount a decorative piece on the top surface of the artificial nail;
the artificial nail comprises: a first edge configured to be disposed near a natural cuticle of the real nail; a second edge opposite to the first edge; a third edge and a fourth edge that are connected between the first edge and the second edge and are spaced apart from each other;
the plurality of guiding grooves comprise a first curved groove and a second curved groove; each of the first curved groove and the second curved groove has one end disposed near the third edge and the other end disposed near the fourth edge; each of the first curved groove and the second curved groove is symmetrical about a centerline of the artificial nail; and the first curved groove has no intersection with the second curved groove.

2. The artificial nail according to claim 1, wherein, the first curved groove has a first apex, the second curved groove has a first apex;
an apex distance is formed between the first apex and the second apex;
an end distance is formed between the one end of the first curved groove and the one end of the second curved groove;
the apex distance is greater than the end distance.

3. The artificial nail according to claim 2, wherein a spacing between the first curved groove and the second curved groove gradually decreases in a direction from the centerline of the artificial nail towards the third edge of the artificial nail.

4. The artificial nail according to claim 1, wherein each of the first curved groove and the second curved groove is curved protruding towards the second edge.

5. The artificial nail according to claim 2, wherein the first apex, the second apex, and a center point of the second edge are located on one straight line coinciding with the centerline of the artificial nail.

6. The artificial nail according to claim 2, wherein the plurality of guiding grooves further comprise a third groove; the third groove extends starting from the first apex, passing through the second apex, to reach a center point of the second edge.

7. The artificial nail according to claim 6, wherein the third groove is extending coinciding with the centerline of the artificial nail.

8. The artificial nail according to claim 1, wherein, each of the plurality of guiding grooves has a groove depth of d and a groove width of b; and 0.05 mm≤d≤0.2 mm, 0.05 mm≤b≤0.2 mm.

9. The artificial nail according to claim 1, wherein the plurality of guiding grooves are used for receiving a nail polish material; a glossiness of a groove wall of each of the plurality of guiding grooves is greater than a glossiness of portions of the top surface of the artificial nail other than the recessed portion.

10. The artificial nail according to claim 9, wherein the portions of the top surface of the artificial nail other than the recessed portion is matte and the groove wall of the guiding groove is glossy.

11. The artificial nail according to claim 9, wherein the top surface of the artificial nail is frosted.

12. The artificial nail according to claim 9, wherein, the artificial nail has a bottom surface opposite to the top surface;
the bottom surface is frosted; and
the bottom surface is configured to be glued to the real nail of the user.
